(19) United States
(12) Patent Application Publication
(10) Pub. No.: US 2016/0272932 A1
(43) Pub. Date: Sep. 22, 2016

(54) Cell culture apparatus with mechanical and temperature control

(71) Applicant: Thomas Allen Precht, Gaithersburg, MD (US)

(72) Inventor: Thomas Allen Precht, Gaithersburg, MD (US)

(21) Appl. No.: 14/880,712

(22) Filed: Oct. 12, 2015

Related U.S. Application Data
(60) Provisional application No. 62/133,910, filed on Mar. 16, 2015.

(51) Int. Cl. C12M 1/00; C12M 1/02; C12M 3/06

(52) U.S. Cl. CPC C12M 41/14; C12M 27/16; C12M 41/18

(57) ABSTRACT

A cell culture controlling device is able to precisely control a cell culture's temperature and physical position. It contains a thermoelectric unit, a heat transfer medium, a controller, a pitch actuation unit, a medium temperature sensor, an ambient temperature sensor, a position sensor, a user interface, a powder supply and an alarm. The cell culture controlling device is able to maintain a cell culture temperature within a range from −10° C. to 70° C. with an increment of 1° C. and maintain the physical position of a cell culture container within a range from 0° to 45° with an increment of 1°. In this device, the thermoelectric unit is a Peltier solid state cooler, which allows an easy and accurate temperature control.

The current application claims a priority to the U.S. Provisional Patent Application Ser. No. 62/133,910, filed on Mar. 16, 2015.

### FIELD OF THE INVENTION

The present invention relates generally to cell culture. More specifically, it is a cell culture device with an automatic control for temperature and movement.

### BACKGROUND OF THE INVENTION

Cell culture is the process by which cells are grown under controlled conditions, generally outside of their natural environment. In practice, cell culture refers to the growth of cells derived from multi-cellular eukaryotes, especially animal cells, outside of the organism. However, there are also cultures of plants, fungi, and microbes, including viruses, bacteria and protists. The laboratory technique of maintaining live cell lines (a population of cells derived from a single cell and containing the same genetic makeup) separated from their original tissue source is a fundamental basis of many biological studies.

Biological research is the cornerstone of all medical discoveries and potential treatments or cures for human disease. Almost all biological research relating to human disease starts in the laboratory with manipulations of individual cells in culture and eventually moves into the clinical setting where human patients can be tested. This basic research on cultured cells, whether they are primary cells dissected from whole animals or secondary cells derived from immortalized tumor lines, relies on maintaining near perfect viability throughout any given experiment.

In most cell based biological, cellular or molecular experiments, maintenance of biological material is assured by keeping cells/tissue cold during manipulations, but also by minimizing the time that sensitive cellular material, such as DNA, RNA, enzymes and protein factors, is exposed to the environment. A common practice is to use an ice bucket that contains ice or a mixture of ice and water to maintain the near 4° C. temperature necessary for structure or activity preservation.

Most buckets are just a conventional plastic or rubber bucket, which are unwieldy, large and not ergonomic. Moreover, it is desirable to perform most cellular assays in the sterile environment of a laminar flow hood. Many large ice buckets prevent an efficient use of the hood for this purpose simply because they take up too much space and don't allow the researcher enough room to operate effectively. More importantly, manipulation of a culture dish during experiments often requires one hand to move the cell culture dish, and the other hand to operate a pipette or other apparatus. When moving the dish to various angles, the dish or a part of the dish frequently is no longer in contact with the ice, thereby allowing some cells to warm up slightly over time or to cool and then warm repeatedly, which is also detrimental to cell viability. In general the researcher's two hands could be used for other purposes (holding a pipette and a tube for example) during cell culture experiments, if there were a device that could heat and cool the dish, and at the same time could tilt the dish to various angles while consistently maintaining temperature. In addition to tilting to certain angles, this device is also able to continuously “rock” the dish back and forth, at controllable frequencies, all while maintaining temperature. The difference between the tilting function and rocking function is as follows, when a tilting function is carried out, the device tilts to a certain angle and then remains in that position for a pre-determined period of time without further movement; whereas when a rocking function is carried out, the device continuously rocks back and forth at a pre-determined frequency. A device of this nature provides the researcher more control over variables which can affect cell viability.

Moreover, a convenient heating and cooling technology is already available in practice and thus can be applied to the field of cell culture. Up to date, thermal management is possible in a broad range of scales and physical spaces, and at a low cost. Thermoelectric units/coolers (TECs), or Peltier devices as they are also known, use current from a power supply to create a temperature differential (the Peltier effect).

Depending on the polarity of the current, a thin sandwich of ceramics and semiconductors creates a cold side and a hot side in a Peltier device. The appeal of these devices is that they can rapidly change temperature, have a long reliable lifespan, have no moving parts (no noise), contain no hazardous materials (no refrigerants), can be precisely controlled, and with reasonable power consumption. Such a device can attain temperature ranges as great as −10° C. to 70° C., which covers the temperature ranges for most cell culture experiments. One caveat of such a device is that if you are cooling on one side, you must dissipate the heat produced on the opposing side with heat sinks and fans.

In light of the foregoing, there is a need for a convenient apparatus to automatically control the temperature of cell cultures, to improve cell based biological experiments. Therefore, one objective of the present invention is to provide a convenient heating and cooling device that fits a majority of culture dish sizes. It enables a researcher to move a dish to another place, tilt a dish to various angles, or rock the dish back and forth thereby aiding in certain cellular manipulations (scraping, triturating, washing, incubating etc . . . ) all while maintaining a constant temperature. A device of this nature will increase efficiency, improve control over variables, enhance productivity, and save time. This device can be easily moved from the bench top to the hood to the incubator, can be sterilized after each use, and can be used for both cooling and heating. This device will be inexpensive to produce given the small size and relatively simple mechanisms supporting it.

### BRIEF DESCRIPTION OF THE DRAWINGS

FIG. 1 is a block diagram of the cell culture device according to the present invention.

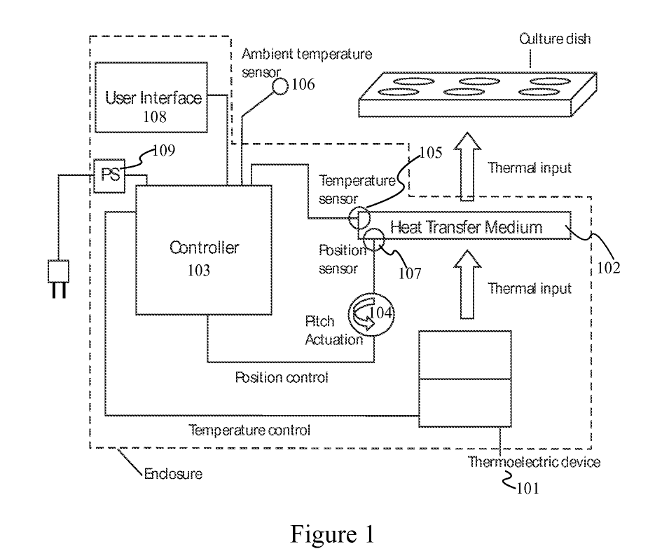

FIG. 2 is a front view of the present invention, in which the culture dish is modeled after a Corning 6-well polystyrene dish.

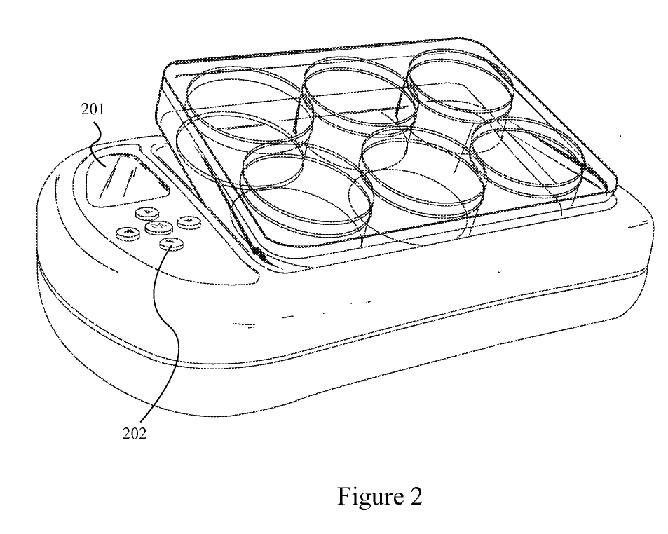

### DETAIL DESCRIPTIONS OF THE INVENTION

All illustrations of the drawings are for the purpose of describing selected versions of the present invention and are not intended to limit the scope of the present invention.

The present invention is a convenient cell culture device with functions to precisely control temperature and mechanical movement such as tilting for the cultured cells. The originality of the cell culture manipulation device is illustrated by combining two very basic elements, the prime mover or motion control and thermoelectric device, which alone are commonplace mechanisms, into an instrument that can fulfill the needs of any cell culture researcher. In addition to combining simple devices for a novel use, the eventual product that is created will have a strong emphasis on user interface and ease of use.

The present invention contains a temperature sensor and a position sensor 107, which allow the controller 103 to manipulate or maintain the desired physical position and temperature; while the ambient temperature sensor will help more precisely control the thermoelectric device's output. In addition, the present invention includes other important components, such as a thermoelectric device (Peltier device), the controller, a user interface 108, a power supply 109, and an enclosure.

The present invention includes four primary components as set forth below. Along with other components, such as various sensors, a power supply and user interface, these primary components are able to precisely control the physical position and temperature of a cell culture container, such as a cell culture dish or plate.

A thermoelectric unit 101 is a Peltier solid state cooler in the current embodiment, but could be any type of device that provides heating and cooling capability.

A heat transfer medium 102 is a custom base plate made of aluminum or other high thermal conductivity material that is formed to provide maximum mating surfaces to the shape of the culture dish. The base plate will have an appropriate coating to prevent contamination from biological materials used in the culture dish.

A controller, with temperature, position and timer sensors plus display 201 and menu buttons for a user interface provides the necessary feedback loop for correct operation of the device such that the temperature of the media can be controlled within a target temperature and appropriate tolerance. It also includes sensors necessary for position measurement of the base plate when the culture dish is to be tilted such that it will move the culture media to a position in the culture dish so a researcher can transfer or otherwise assay the biological materials in the media.

A pitch actuation unit 104 is a motor and mechanism such that it can tilt the base plate by 1° increments from 0° to 45°. The mechanism can also continuously tilt the dish from 0° to 45°, while controlling the acceleration and deceleration of the dish, and the frequency of the tilting. Via continuous tilting, at variable frequencies within the range of 0.5 Hz (cycles/sec) to 2 Hz, the mechanism creates a rocking motion, moving both the dish and the liquid contents of the dish.

In addition to the above mentioned components, the present invention also contains a few sensors, which mainly include a position sensor, a medium temperature sensor 105 and an ambient temperature sensor 106. The position sensor functions to sense the exact position of the heat transfer medium, so as to accurately control the position or tilting movement of the heat transfer medium, as well the cell culture container thereon. The medium temperature sensor and the ambient temperature sensor are able to sense the temperature of the heat transfer medium and the ambient temperature, respectively. The sensing temperature information will further be sent to the controller, so as to attain an accurate temperature for the cell culture conducted on the present invention.

The interface includes a key pad that contains a few buttons and a display, preferably an LCD display, which show certain information or controlling command. The instructions may be inputted via a key pad or through a touch pad 202.

In addition, the present invention also includes a power supply and an enclosure. The power supply functions to provide power for the entire device of the present invention. The enclosure covers key components of the present invention inside, so as to provide a clean design and protection of these components from external interferences.

In an operation, a user places a culture dish in the base plate, such that it nests within the special formed features of the device to give maximum thermal contact with the dish. The medium temperature sensor, such as a thermistor, is connected into the base plate and reads the temperature of the base plate. There may be a plurality of temperature sensors that are placed on the base plate and make contact with the culture dish such that the temperature of the culture dish is known. The user then operates the menu keys to program a desired temperature and time sequence, which can include multiple temperature profiles and tilt angle protocols that the culture media will be set to. An alarm is also included to signal the user when the culture dish has reached target temperature or time.

The present invention is able to provide a convenient operation and accurate temperature control for a cell culture process. Up to date, there are few closed loop feedback thermal control devices that provide temperature control of culture media on the market at this time. This device is able to provide accurate control of culture media temperature, without intervention of the researcher; provide rapid temperature changes, faster than typical “ice bucket” methods, in particular when wanting to have media at increased temperature such as 37° C.; provide tilting of the culture dish for ease of transfer of culture media; provide rocking of the culture dish; provide easy to use menu with display for setting temperature, tilt and time parameters for a researcher's experiments. The device according to the present invention significantly increases the efficiency of a researcher, both with respect to time required for the researcher to “babysit” the experiments and with potentially increased yield of cellular materials in the culture media.

Although the invention has been explained in relation to its preferred embodiment, it is to be understood that many other possible modifications and variations can be made without departing from the spirit and scope of the invention as herein described.

What is claimed is:

1. A cell culture controlling device, comprising
a thermoelectric unit;
a heat transfer medium;
a controller;
a pitch actuation unit;
a medium temperature sensor;
an ambient temperature sensor;
a position sensor;
a user interface;
a power supply;
the medium temperature sensor, the ambient temperature sensor and the position sensor communicating with the controller;
the power supply connecting to the controller;
the thermoelectric unit being in physical contact with the heat transfer medium;
the controller communicating with the thermoelectric unit; and
and the user interface communicating with the controller.

2. The cell culture controlling device as set forth in claim 1, comprising:
the user interface comprising a key pad and a display;
a user sending an instruction through the key pad;
the instruction being displayed on the display; and
a feedback or an operation state being shown on the display.

3. The cell culture controlling device as set forth in claim 2, comprising:
the operation state comprising an operation temperature and an operation position.

4. The cell culture controlling device as set forth in claim 2, comprising:
the display is an LCD display.

5. The cell culture controlling device as set forth in claim 3, comprising:
the operation temperature is within a range from −10° C. to 70° C. with an increment of 1° C.

6. The cell culture controlling device as set forth in claim 3, comprising:
the operation position is within a range from 0° to 45° with an increment of 1°.

7. The cell culture controlling device as set forth in claim 1, comprising:
the thermoelectric unit is a Peltier solid state cooler.

8. The cell culture controlling device as set forth in claim 1, wherein the device is tiltable to a predetermined angle and remains in the angle for a pre-determined time period.

9. The cell culture controlling device as set forth in claim 1, wherein the device is rockable back and forth at a pre-determined frequency.

10. The cell culture controlling device as set forth in claim 1, comprising:
the medium temperature sensor being in contact with the heat transfer medium;
the medium temperature sensor sensing a temperature of the heat transfer medium;
the position sensor being in contact with the heat transfer medium; and
the position sensor sensing a physical position of the heat transfer medium.

11. The cell culture controlling device as set forth in claim 1, comprising:
the medium temperature sensor is a thermistor.

12. The cell culture controlling device as set forth in claim 1, further comprising an alarm, the alarm alerting a user when the heat transfer medium has reached a target temperature or a target culture time period.

13. A cell culture controlling device, comprising
a thermoelectric unit;
a heat transfer medium;
a controller;
a pitch actuation unit;
a medium temperature sensor;
an ambient temperature sensor;
a position sensor;
a user interface;
a power supply;
an alarm;
the medium temperature sensor, the ambient temperature sensor and the position sensor communicating with the controller;
the power supply connecting to the controller;
the thermoelectric unit being in physical contact with the heat transfer medium;
the controller communicating with the thermoelectric unit;
and the user interface communicating with the controller;
the medium temperature sensor being in contact with the heat transfer medium;
the medium temperature sensor sensing a temperature of the heat transfer medium;
the position sensor being in contact with the heat transfer medium;
the position sensor sensing a physical position of the heat transfer medium; and
the alarm alerting a user when the heat transfer medium has reached a target temperature or a target culture time period.

14. The cell culture controlling device as set forth in claim 13, comprising:
the user interface comprising a key pad and a display;
a user sending an instruction through the key pad;
the instruction being displayed on the display;
a feedback or an operation state being shown on the display;
the operation state comprising an operation temperature and an operation position; and
the display is an LCD display.

15. The cell culture controlling device as set forth in claim 14, comprising:
the operation temperature is within a range from −10° C. to 70° C. with an increment of 1° C.; and
the operation position is within a range from 0° to 45° with an increment of 1°.

16. The cell culture controlling device as set forth in claim 13, comprising:
the thermoelectric unit is a Peltier solid state cooler.

17. The cell culture controlling device as set forth in claim 13, comprising:
the thermoelectric unit is a Carnot cycle heat pump.

18. The cell culture controlling device as set forth in claim 13, comprising:
the medium temperature sensor is a thermistor.

19. The cell culture controlling device as set forth in claim 13, wherein the device is tiltable to a predetermined angle and remains in the angle for a pre-determined time period.

20. The cell culture controlling device as set forth in claim 13, wherein the device is rockable back and forth at a pre-determined frequency.
